(19) United States
(12) Patent Application Publication
(10) Pub. No.: US 2014/0206058 A1
(43) Pub. Date: Jul. 24, 2014

(54) SYSTEMS AND METHODS FOR IMPROVING STILLAGE

(71) Applicants: Jacob P. Tewalt, Sioux Falls, SD (US); Stephen M. Lewis, Sioux Falls, SD (US); Jason Alan Bootsma, Sioux Falls, SD (US)

(72) Inventors: Jacob P. Tewalt, Sioux Falls, SD (US); Stephen M. Lewis, Sioux Falls, SD (US); Jason Alan Bootsma, Sioux Falls, SD (US)

(73) Assignee: POET RESEARCH, INC., Sioux Falls, SD (US)

(21) Appl. No.: 14/004,136

(22) PCT Filed: Mar. 8, 2012
(86) PCT No.: PCT/US12/28311

§ 371 Date: Nov. 26, 2013

Related U.S. Application Data
(60) Provisional application No. 61/450,228, filed on Mar. 8, 2011.

(51) Int. Cl. C12P 1/02

(52) U.S. Cl. CPC C12P 1/02
USPC 435/171; 435/289.1

(57) ABSTRACT

Systems and methods for improving stillage are disclosed. Stillage may include either whole stillage or thin stillage. The system includes taking the stillage and placing it within a bioreactor with an inoculation of fungi. The fungi may include any of Aspergillus niger, Phanerochaete chrysosporium and Yarrowia lipolytica. The fungi and stillage broth is then subjected to fermentation which removes solubles and particulates from the stillage. The fungi generate a biomass material that may be collected and dried for use as a nutritional supplement or other purpose. The remaining liquid is a clarified, treated stillage suitable for a variety of downstream applications, including being used as a backset in an ethanol production facility.

### CROSS-REFERENCE TO RELATED APPLICATIONS

This application is a U.S. national stage filing of Patent Cooperation Treaty (PCT) application serial number PCT/US2012/028311 entitled “SYSTEMS AND METHODS FOR IMPROVING STILLAGE” filed on Mar. 8, 2012, which claims the benefit of U.S. Provisional Application Ser. No. 61/450,228, filed Mar. 8, 2011, and entitled “SYSTEMS AND METHODS FOR IMPROVING STILLAGE”. The entireties of the aforementioned applications are herein incorporated by reference.

### FIELD

The subject disclosure relates to systems and methods for clarification of thin and whole stillage in an ethanol production facility using microorganisms.

### BACKGROUND

Ethanol traditionally has been produced from grain-based feedstocks (e.g., corn, sorghum/milo, barley, wheat, soybeans, etc.) or from sugar (e.g., sugar cane, sugar beets, etc.).

In a conventional ethanol plant, corn, sugar cane, other grain, beets, or other plants are used as a feedstock and ethanol is produced from starch contained within the corn, or other plant feedstock. In the case of a corn facility, corn kernels are cleaned and milled to prepare starch-containing material for processing. Corn kernels can also be fractionated to separate the starch-containing material (e.g., endosperm) from other matter (such as fiber and germ). Initial treatment of the feedstock varies by feedstock type. Generally, however, the starch and sugar contained in the plant material is extracted using a combination of mechanical and chemical means.

The starch-containing material is slurried with water and liquefied to facilitate saccharification, where the starch is converted into sugar (e.g., glucose), and fermentation, where the sugar is converted by an ethanologen (e.g., yeast) into ethanol. The fermentation product is beer, which comprises a liquid component, including ethanol, water, and soluble components, and a solids component, including unfermented particulate matter (among other things). The fermentation product is sent to a distillation system where the fermentation product is distilled and dehydrated into ethanol. The residual matter (e.g., whole stillage) comprises water, soluble components, oil, and unfermented solids (e.g., the solids component of the beer with substantially all ethanol removed, which can be dried into dried distillers grains (DDG) and sold, for example, as an animal feed product). Other co-products (e.g., syrup and oil contained in the syrup), can also be recovered from the whole stillage.

In a typical ethanol plant, a massive volume of whole stillage is generally produced. For example, for a midsize ethanol plant the amount of whole stillage produced can be near 13.4 gallons per bushel of corn processed. Roughly a third of the corn feedstock is present in the whole stillage as dissolved organics and solids. The stillage contains almost 90% water. Whole stillage is responsible for a substantial portion of the wastewater generated by ethanol plants. The financial cost of the water, its treatment and disposal (typically through evaporation) can be very large. Additionally, the use and disposal of such large amounts of wastewater may have a negative impact upon local watersheds and the environment as a whole.

In the interest of improving efficiencies of ethanol plants, whole stillage is often separated into two components: a solid component and a liquid component. Separation may be performed using centrifugation, or filter and press. The solid component may be dried to generate dried distillers grain (DDG) which is sold as animal feed. DDG is low in essential amino acids, particularly lysine, which may limit its use. The liquid component, known as thin stillage, may be dried and used to increase the protein content of DDG to make DDGS (Distillers Dried Grains with Solubles). This process requires the drying of a large amount of water, which is very energy intensive and costly. Thin stillage may also be recycled into the plant, such as for replacement of some portion of the water used during fermentation (fermentation backset). Using thin stillage as a fermentation backset reduces the total water that needs to be evaporated; however, under current technologies, there is a limit to the percentage of thin stillage that may be recycled into the fermentation, as the dissolved solids in the thin stillage tend to inhibit the fermentation process.

A number of methods have been developed for the treatment of thin stillage in order to reduce the cost and burden of disposal. These treatment methods include microfiltration of the thin stillage, chemical treatments, and biological treatments. The biological treatments include the application of fungal spores to thin stillage in order to clean the stillage, as is discussed in U.S. Patent Publication No. 2008/0153149 by Johannes Van Leeuwen et al. These methods of thin stillage treatment are directed to the cleaning of water so that it may be utilized in a broader range of downstream uses (such as cleaning, backset and fire extinguishing). While these methods function to remove dissolved organics within thin stillage, the resulting treated stillage is basically reduced to a low-grade water.

### SUMMARY

The disclosed aspects relate to systems and methods for improving the quality of stillage from an ethanol production facility. Such systems and methods can convert a low value waste product of the ethanol production process into a valuable co-product, thereby increasing revenue and decreasing waste from ethanol plants.

Stillage may include either whole stillage or thin stillage. The system includes taking the stillage and placing it within a bioreactor with an inoculation of fungi. The fungi may include any of Aspergillus niger, Phanerochaete chrysosporium and the yeast Yarrowia lipolytica. The fungi and stillage broth is then subjected to fermentation.

The fermented broth removes solubles and particulates from the stillage. The fungi generate a biomass material that can be collected and dried for use as a nutritional supplement or for other purposes. The remaining liquid is a clarified, treated stillage suitable for a variety of downstream applications. In addition to being clarified by the fungal fermentation, the fungal cells also produce extracellular enzymes which can increase the efficiency of ethanol fermentation when the treated stillage is used as a backset in an ethanol production facility.

The fermentation process is performed at about 20 to 40° C. and at a pH of about 4 to 6. The inoculation of the fungi may include inoculating either spores and/or a cell culture. Lastly, the fermentation can be agitated and/or aerated. Often fermentation is performed within an airlift bioreactor, or similar bioreactor.

Note that the various features of the various aspects described above may be practiced alone or in combination. These and other features of the aspects disclosed herein will be described in more detail below in the detailed description and in conjunction with the following figures.

### DESCRIPTION OF THE DRAWINGS

In order that the disclosed aspects may be more clearly ascertained, some embodiments will now be described, by way of example, with reference to the accompanying drawings, in which:

FIG. 1 is a perspective view of a biorefinery comprising an ethanol production facility, in accordance with some embodiments;

FIGS. 2A and 2B are process flow diagrams illustrating examples of ethanol production processes from corn to ethanol, in accordance with some embodiments;

FIGS. 3A and 3B are schematic diagrams illustrating examples of systems for treatment to improve stillage, in accordance with some embodiments;

FIGS. 4A and 4B are process flow diagrams illustrating examples of methods for treatment to improve stillage, in accordance with some embodiments;

FIGS. 5 to 8 are example graph diagrams illustrating compositional results of the growth of fungal material on stillage, in accordance with some embodiments;

FIG. 9 is an example graph illustrating fermentation efficiency based upon enzyme loading concentration and backset makeup, in accordance with some embodiments;

FIG. 10 is an example graph illustrating biomass saccharification efficiency based upon backset makeup, in accordance with some embodiments;

FIG. 11 is an example graph illustrating protein production by fungus, in accordance with some embodiments;

TABLE 1 lists the mass balance composition of thin stillage, fermentation broth and resulting liquid and solid compositions, in accordance with some embodiments for Yarrowia lipolytica;

TABLE 2 lists the percent solids composition for the fermentation broth and resulting liquid and solid fractions for Aspergillus niger;

TABLE 3 indicates the amount of single cell protein generated per bushel of corn, in accordance with some embodiments; and

TABLE 4 lists the nutritional composition of Aspergillus niger solid fractions, in accordance with some embodiments.

### DESCRIPTION OF THE EMBODIMENTS

The various aspects will now be described in detail with reference to several embodiments thereof as illustrated in the accompanying drawings. In the following description, numerous specific details are set forth in order to provide a thorough understanding of embodiments of the various aspects. It will be apparent, however, to one skilled in the art, that embodiments may be practiced without some or all of these specific details. In other instances, well known process steps and/or structures have not been described in detail in order to not unnecessarily obscure the disclosed aspects. The features and advantages of embodiments may be better understood with reference to the drawings and discussions that follow.

Given the nutrient content of stillage and the need for water in the fermentation of beer, to the various aspects provide for systems and methods that improve stillage for use in backset and as a nutritional supplement in a cost effective manner. Such systems and methods can provide a substantial reduction in fermentation costs, increased revenue for nutritional co-products, and a lower impact on the environment.

The aspects disclosed herein relate to systems and methods for improving stillage from an ethanol production plant. Ethanol plants generate large quantities of stillage as a largely waste product. Stillage is generally a low value co-product that requires substantial energy to dry into solubles for addition to distillers dried grains, or must be disposed of in some other manner. The disclosed aspects provide a means to substantially improve the quality and value of stillage by generating single cell protein co-products and improve clarity and quality of the treated stillage. Higher quality of the stillage can increase its range of applicable use to virtually any water dependent process, including backset for fermentation, or hydrolysis of biomass in a biorefinery.

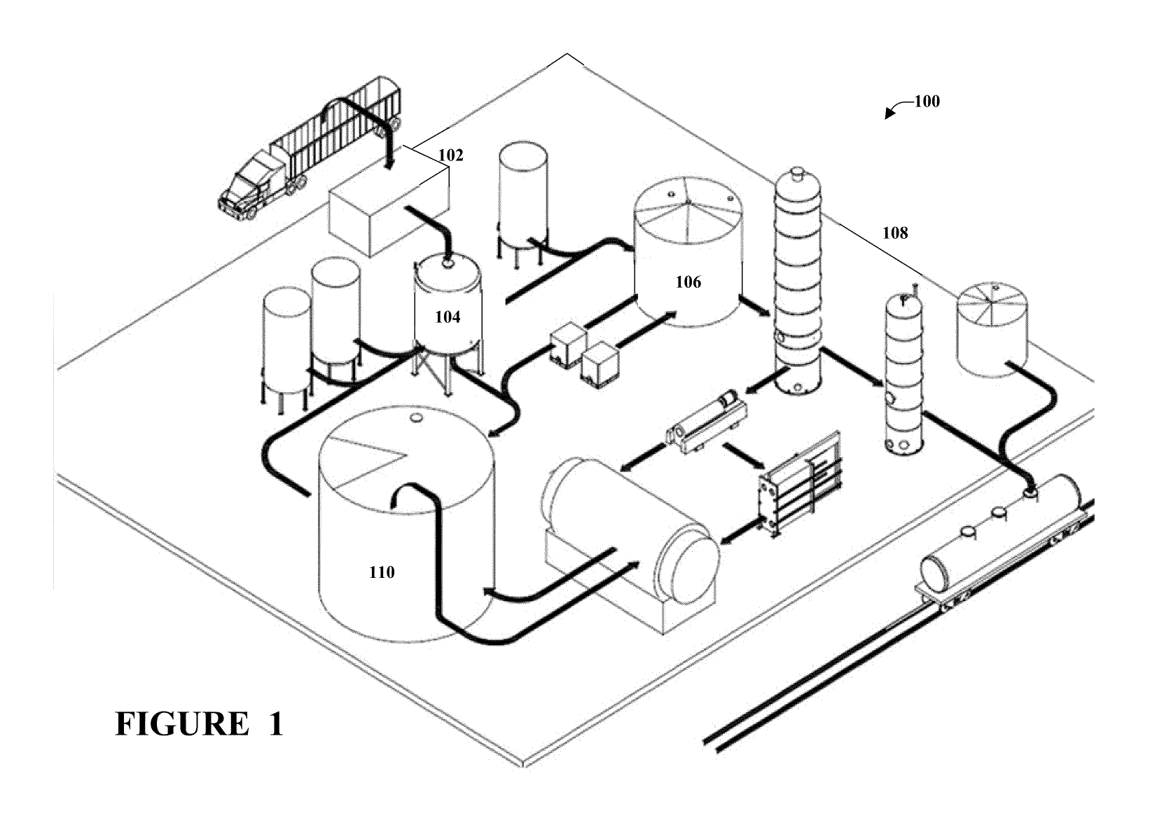

Referring to FIG. 1, an example biorefinery 100 comprising an ethanol production facility configured to produce ethanol from corn is illustrated. The example biorefinery 100 comprises an area 102 where corn (or other suitable material including, but not limited to, biomass, sugars, and other starch products) is delivered and prepared to be supplied to the ethanol production facility. The ethanol production facility comprises apparatus 104 for preparation and treatment (e.g., milling) of the corn into corn flour suitable for fermentation into fermentation product in a fermentation system 106. The ethanol production facility comprises a distillation system 108 in which the fermentation product is distilled and dehydrated into ethanol. The biorefinery may also comprise, in some embodiments, a by-product treatment system (shown as comprising a centrifuge, a dryer, and an evaporator).

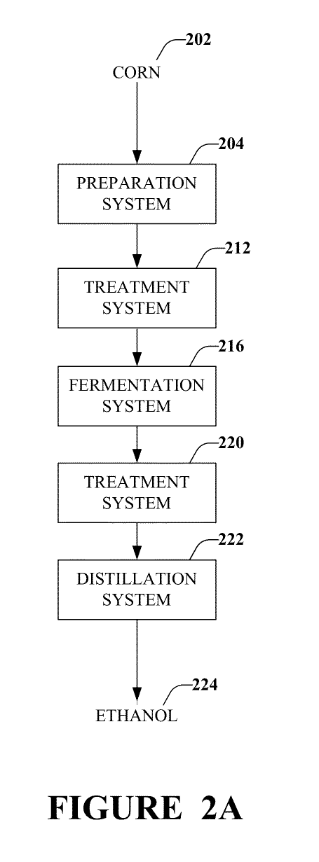

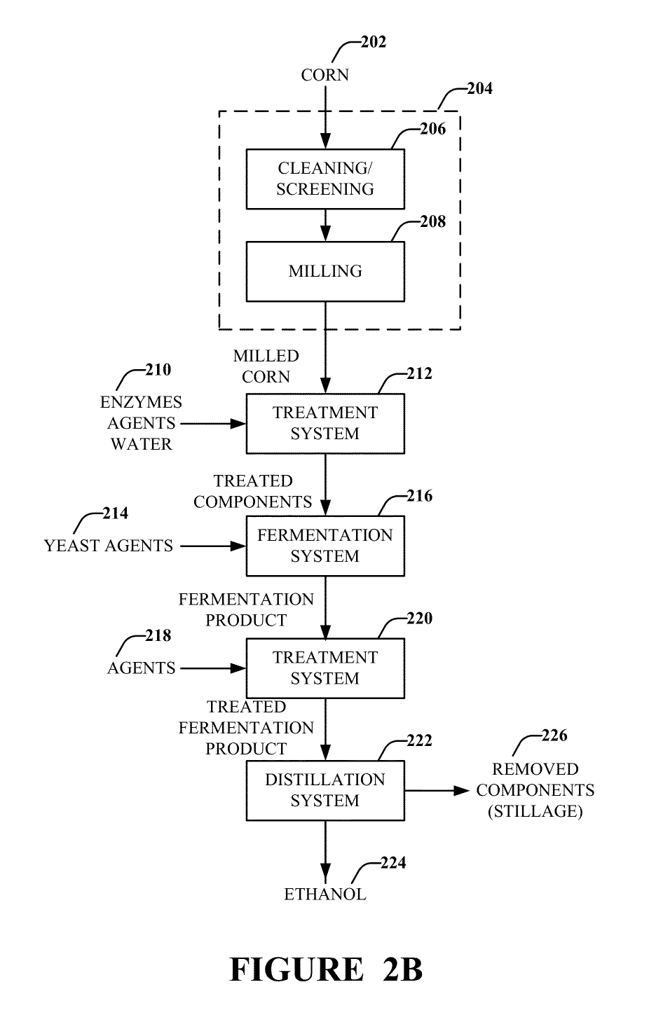

Referring to FIGS. 2A and 2B, in an ethanol production process, corn 202 (or other suitable feed material) may be prepared for further treatment in a preparation system 204. As illustrated in FIG. 2B, the preparation system 204 may comprise cleaning or screening 206 to remove foreign material, such as rocks, dirt, sand, pieces of corn cobs and stalk, and other unfermentable material (e.g., removed components). After cleaning or screening 206, the particle size of corn may be reduced by milling 208 to facilitate further processing. The corn kernels may also be fractionated into starch-containing endosperm, fiber, and germ, in accordance with some embodiments. The milled corn or endosperm is slurried with water, enzymes and agents 210 to facilitate the conversion of starch into sugar (e.g. glucose), such as in a first treatment system 212. The sugar (e.g., treated component) is converted into ethanol by an ethanologen (e.g. yeast or other agents 214) in a fermentation system 216. The product of fermentation (fermentation product) is beer, which comprises a liquid component, including ethanol and water and soluble components, and a solids component, including unfermented particulate matter (among other things). The fermentation product may be treated with agents 218 in a second treatment system 220. The treated fermentation product is sent to a distillation system 222. In the distillation system 222, the (treated) fermentation product is distilled and dehydrated into ethanol 224. In some embodiments, the removed components 226 (e.g., whole stillage), which comprise water, soluble components, oil and unfermented solids (e.g., the solids component of the beer with substantially all ethanol removed), may be dried into dried distillers grains (DDG) in a third treatment system (where the removed components may be treated with agents) and sold as an animal feed product. Other co-products, for example, syrup (and oil contained in the syrup), may also be recovered from the stillage.

The thin stillage, that results when solids are removed from the whole stillage, can be used as a backset during the fermentation process and can also be used to increase the protein content of DDGS (Distillers Dried Grains with Solubles). However, dissolved solids that are present in the thin stillage can inhibit the fermentation process and decrease the efficiency of ethanol production. Furthermore, the addition of thin stillage to DDGS requires costly evaporation processes that increase the DDGS production cost. Disclosed herein are systems and methods for using natural fungal processes to improve thin stillage use in at least four manners: 1) reduction of dissolved and total solids, 2) an increased amount of enzymes produced in the backset, 3) single-cell protein production for a value added co-product, and 4) a reduction in energy costs associated with the drying of thin stillage. Previous research into the treatment of thin stillage with fungi has focused on Rhizopus and Aspergilli strains, but the feasibility of numerous other strains has not been fully studied until now.

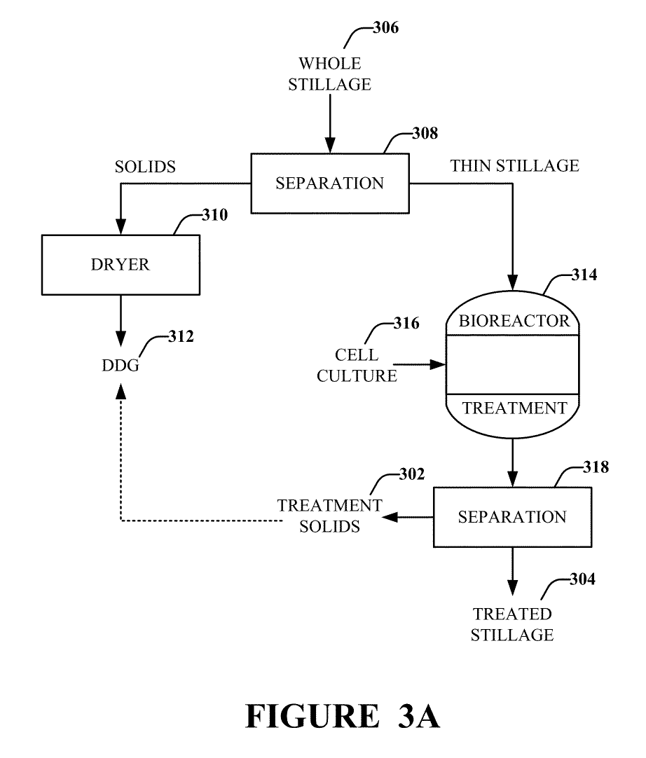

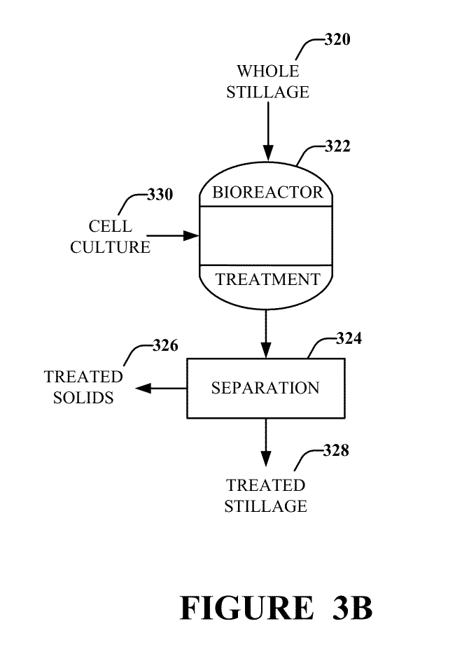

Referring now to FIG. 3A, a first example schematic block diagram of a system for treatment of the removed stillage component in order to produce an improved stillage product is provided. The improved stillage product may yield single cell protein (treatment solids 302) as a valuable co-product as well as treated stillage 304, once separated. The treated stillage 304 can be utilized in a wide range of downstream applications including recycle into the backset of fermentation, use in the hydrolysis of biomass in a cellulosic ethanol production facility, as a wash or other low grade water source, irrigation, or the like.

In this exemplary diagram, whole stillage 306 is provided to a separator 308 for separation into a solids component and a thin stillage component. The separator 308 may include a centrifuge design, screw press and filter, or other system adapted to separating out a fluid component from a solids component. The solids, in some embodiments, may then be provided to a dryer 310 in order to dry into Dried Distillers Grains (DDG 312) for use as a animal feed co-product. The DDG 312 may be further improved through the application of solubles, in some embodiments, to generate DDGS (Dried Distillers Grains with Solubles).

In the exemplary embodiment, the thin stillage that results from the separation of whole stillage 306 may be provided to a bioreactor 314 as a media upon which to grow fungus. The fungus may be provided to the bioreactor 314 as a cell culture 316 inoculation, or via spore inoculation. The bioreactor 314 may be temperature controlled, pH controlled, and include a system of aeration. Proper oxygen content via agitation, aeration or a combination of the two might be necessary for proper fungal propagation, in some embodiments. Thus, a bioreactor can be selected which enables proper aeration of the fungal mixture. Examples of suitable bioreactor designs include airlift bioreactors, for example.

After fungal fermentation in the bioreactor 314, the resulting slurry may be provided to a second separator 318 which separates the liquid treated stillage 304 from the treatment solids 302. Treatment solids 302 can include a cellular mat from the fungus, with additional fermentation solids. The solid resulting from the fungal treatment can be high in single celled proteins, including a high lysine content. This renders the solids as a high value nutritional supplement for animal feed. In some embodiments, the solids may be dried and added to the DDG to generate enhanced DDG with improved nutritional content. In alternate embodiments, the treated solids may instead be utilized as a standalone co-product, such as a milk replacement for young animals.

The treated stillage 304 may likewise be of increased value after treatment. This is due to the fact that through the removal of the solids from the stillage, the treated stillage 304 is now suited for a wider range of uses, including backset in order to offset the water needs of the ethanol plant or other industrial facility. Further, the treated liquids are now clean enough to be utilized for irrigation, cleaning and the like. As a result, less water needs to be consumed by the ethanol facility, and likewise less water requires evaporation. Since less water is evaporated, the ethanol production facility is also able to reduce energy requirements.

In addition to energy savings, the treated stillage 304 may also contain dissolved proteins, which can improve the efficiency of the backset in enzyme dependent processes. For example, treated stillage can increase fermentation efficiency of corn when used as a backset as opposed to fresh water. Likewise, the saccharification of biomass to yield sugars for cellulosic ethanol production can be improved by using treated stillage instead of water.

FIG. 3B illustrates a second schematic block diagram of a system for treatment to improve stillage, in accordance with some embodiments. In this example system, whole stillage 320 is provided directly to a bioreactor 322 without undergoing an initial separation. Such a system can benefit from reduced infrastructural requirements since only a single separator 324 is used to separate solids 326 from liquids 328 post fungal treatment.

Since the solids from the whole stillage 320 are inoculated by a cell culture 330 and/or spore inoculation, the resulting treated solids 326 volume can be much larger. Further, the nutritional value of the treated solids 326 can be reduced as compared to the pure single cell protein mats otherwise produced. However, the resulting treated solids 326, once dried, sill provide an excellent feedstock for animals as an enhanced DDG. Again, the treated stillage 328 can be utilized as a backset, or for any other suitable water balance purpose.

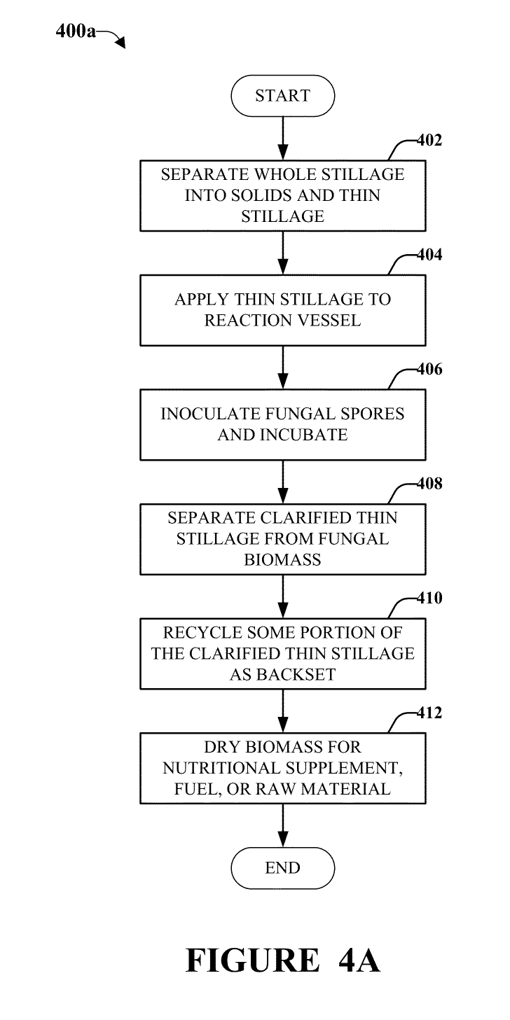

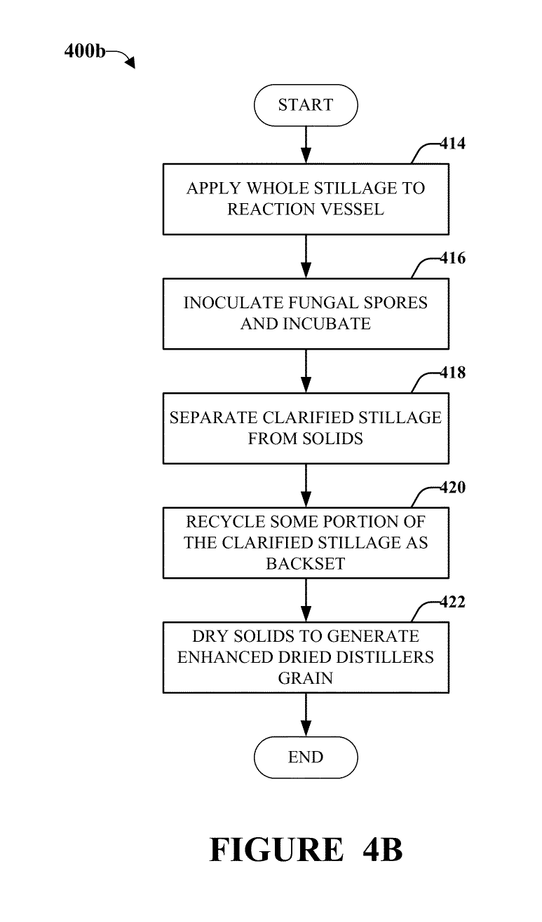

FIG. 4A is a first process flow diagram 400a illustrating an example method for treatment to improve stillage, in accordance with some embodiments. This process flow is suitable for performance on a system such as that illustrated in FIG. 3A. In this process, the whole stillage is separated into the solids and thin stillage (at 402).

The thin stillage is applied to a reaction vessel (at 404) and fungal spores (or cells) are inoculated into the reaction vessel. The vessel is incubated, with aeration, for a suitable period (at 406). The treated thin stillage (treated liquids) are separated from the fungal biomass (treated solids) via centrifugation, filtration or other suitable means (at 408).

At least some portion of the treated thin stillage is recycled as a backset (at 410) into some portion of the process flow of the ethanol plant or other co-located industrial facility. For example, the treated thin stillage generated at a corn ethanol plant could be utilized as a backset makeup for the water used in hydrolysis of biomass in a nearby cellulosic ethanol plant.

The biomass resulting from the fungal incubation can be dried and supplied as a nutritional supplement, fuel or other raw material (at 412). If used as a nutritional supplement, the fungal biomass may be further treated (such as through heating/cooling, milling, or chemical treatments). The fungal biomass may be a standalone nutritional product, or may be added to other nutritional products (such as DDG) in order to increase the nutritional value of the feeds.

FIG. 4B is a second process flow diagram 400b illustrating an example method for treatment to improve stillage, in accordance with some embodiments. This process flow is suitable for performance on a system such as that illustrated in FIG. 3B. In this process, the whole stillage is supplied directly to the reaction vessel (at 414) without separation of the solids prior.

Fungal spores (or cells) are inoculated into the reaction vessel and the vessel is incubated, with aeration, for a suitable period (at 416). The treated thin stillage (treated liquids) are separated from the treated solids via centrifugation, filtration or other suitable means (at 418). At least some portion of the treated thin stillage is recycled as a backset (at 420) into some portion of the process flow of the ethanol plant or other co-located industrial facility. The resulting solids tend to be of larger volume since all of the solids in the whole stillage were incubated with the fungus. Additionally, the nutritional value of these solids tends to be lower than the fungal biomass derived from processing thin stillage, however the solids are still of heightened nutritional value and may be dried to generate an enhanced DDG product (at 422) which may be sold as an animal feed.

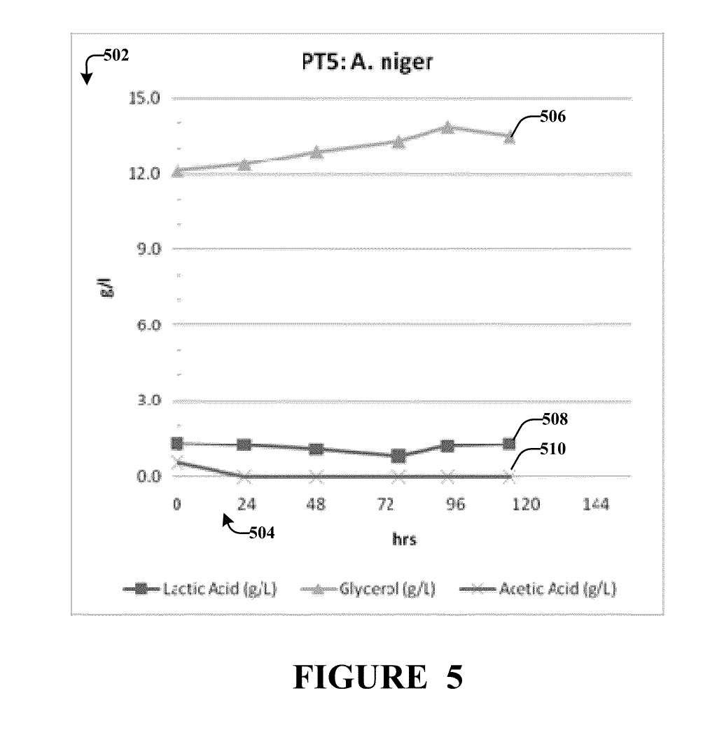

FIGS. 5 to 8 are example graph diagrams illustrating compositional results of the growth of fungal material on stillage, in accordance with some embodiments.

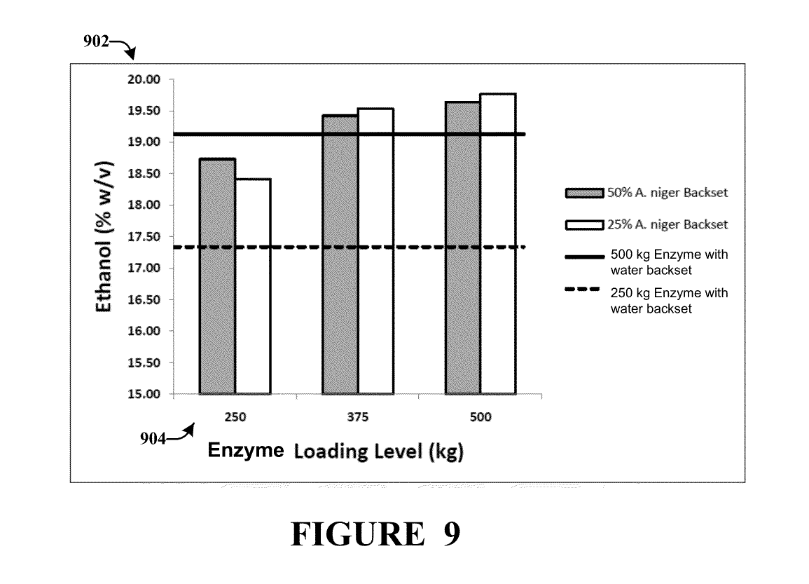

FIG. 9 is an example graph illustrating fermentation efficiency based upon enzyme loading concentration and backset makeup, in accordance with some embodiments.

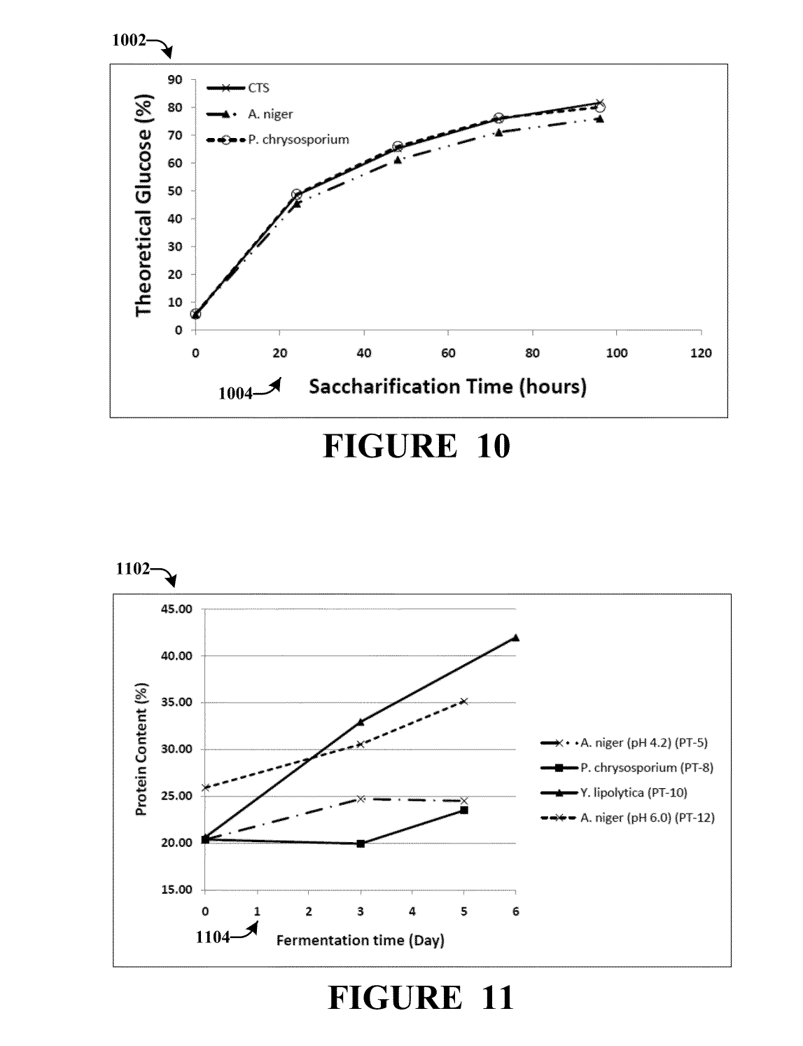

FIG. 10 is an example graph illustrating biomass saccharification efficiency based upon backset makeup, in accordance with some embodiments.

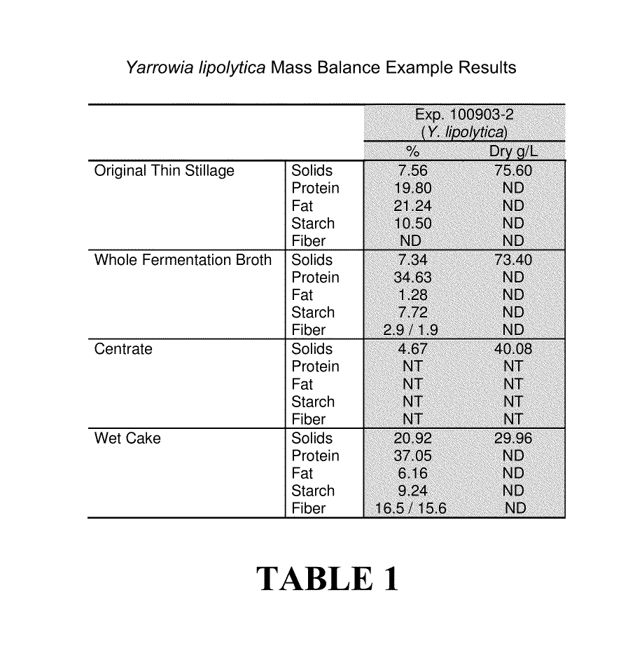

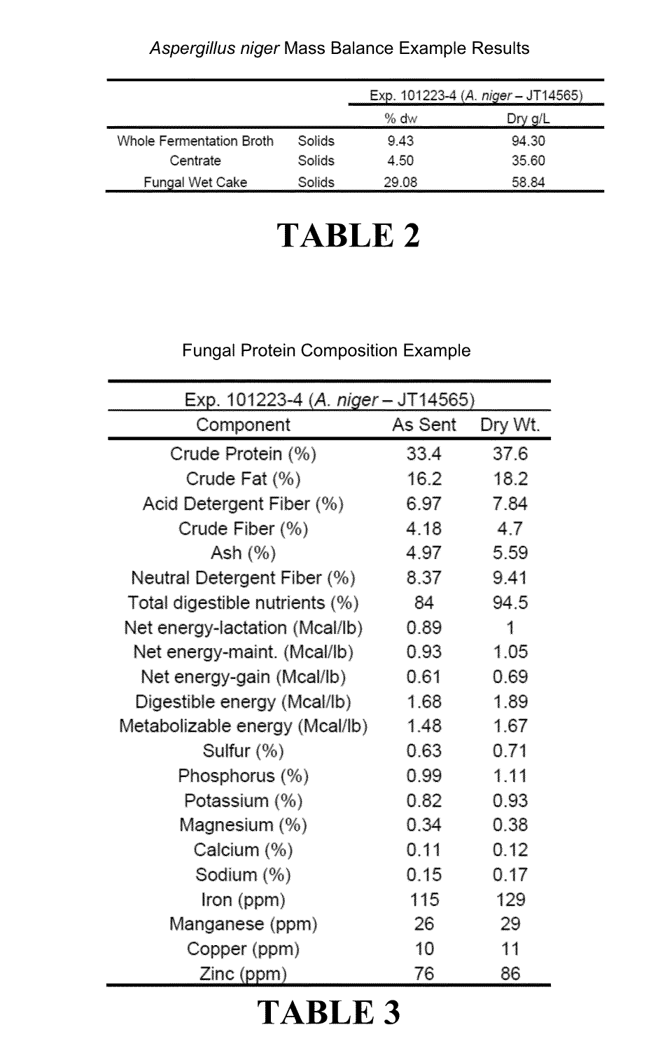

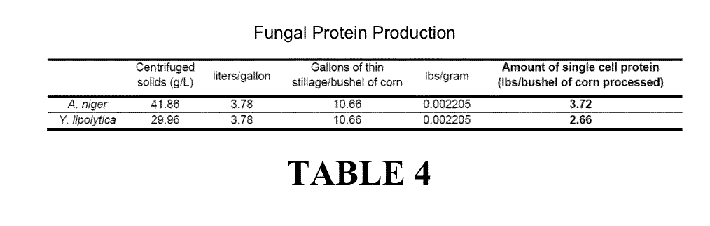

FIG. 11 is an example graph illustrating protein production by fungi, in accordance with some embodiments.

TABLE 1 lists the mass balance composition of thin stillage, fermentation broth and resulting liquid and solid compositions, in accordance with some embodiments for Yarrowia lipolytica.

TABLE 2 lists the percent solids composition for the fermentation broth and resulting liquid and solid fractions for Aspergillus niger.

TABLE 3 indicates amount of single cell protein generated per bushel of corn, in accordance with some embodiments.

TABLE 4 lists the nutritional composition of Aspergillus niger solid fractions, in accordance with some embodiments.

As disclosed herein, an aspect relates to a system for improving stillage. The system comprises a bioreactor configured to receive stillage, a separator configured to remove the fungal biomass from the treated stillage, and a dryer configured to dry the fungal biomass. The bioreactor is further configured to receive an inoculation of a fungi. Further, the bioreactor is configured to ferment the fungi and stillage broth to generate a fungal biomass and a treated stillage. The fungi is at least one of Aspergillus niger, Phanerochaete chrysosporium and Yarrowia lipolytica.

In an aspect, the fungi and stillage broth is maintained at a temperature at about 20 to 40° C. The fungi and stillage broth can be maintained at a pH at about 4 to 6, according to an aspect.

In some aspects, the inoculation of the fungi includes at least one of inoculating spores and inoculating a cell culture. The system, in an aspect, further comprises piping configured to direct the treated stillage to a fermentation system as backset. In some aspects, the bioreactor is agitated or aerated. The bioreactor can be an airlift type bioreactor. The stillage can include whole stillage or thin stillage.

Another aspect relates to a method for improving stillage. The method comprises receiving stillage, inoculating the stillage with a fungi to generate a broth, and fermenting the broth to generate a fungal biomass and a treated stillage. The method also comprises removing the fungal biomass from the treated stillage and drying the fungal biomass. The fungi is at least one of Aspergillus niger, Phanerochaete chrysosporium and Yarrowia lipolytica.

In some aspects, the fermenting comprises maintaining the broth at a temperature at about 20 to 40° C. during the fermenting. The fermenting comprises maintaining the broth at a pH at about 4 to 6 during the fermenting, according to some aspects. The inoculating the stillage with the fungi can comprise at least one of inoculating spores and inoculating a cell culture.

The method, according to some aspects, further comprises directing the treated stillage to a fermentation system as a backset.

In accordance with some aspects, the fermenting comprises agitating the broth during the fermenting. In some aspects, the fermenting comprises aerating the broth during the fermenting. In other aspects, the fermenting comprises fermenting the broth in an airlift type bioreactor.

According to some aspects, the receiving comprises receiving stillage that comprises whole stillage. In accordance with some aspects, the receiving comprises receiving stillage that comprises thin stillage.

A series of limited examples were conducted according to an exemplary embodiment of the system (as shown in FIG. 3A) in an effort to determine suitable apparatus and operating conditions for the treatment of lignocellulosic hydrolysate to mitigate fermentation inhibitors. The following examples are intended to provide clarity to some embodiments of systems and means of operation; given the limited nature of these examples, they do not limit the scope of the invention.

### Example 1

In this example experiment fungal fermentations were performed using various fungal strains in thin stillage collected from an ethanol plant. The fermentations were run for a total of 5-6 days at 30-40° C. and at a pH of 4.5 or 6.0. The ability of each strain to reduce dissolved solids was tested using high performance liquid chromatography (HPLC) for analysis of sugars, acids and sugar alcohols as detailed in FIGS. 5 to 8.

FIG. 5 illustrates the results for the strain A. niger. For this experiment, 20 liters of broth was fermented in a 30 liter fermentor vessel. The pH was maintained at 4.5 and temperature was maintained at 30 degrees Celsius. Samples of liquids and solids were taken each day over the fermentation period. Results from the analysis of the samples are shown with grams per liter of each component illustrated on the vertical axis, at 502, versus the length of fermentation, as indicated at 504. Glycerol 506, lactic acid 508 and acetic acid 510 are each plotted. For A.niger the glycerol content increased slowly as a function of fermentation length. As illustrated, acetic acid is reduced within the first 24 hours of fungal fermentation. Levels of lactic acid appear to remain steady over the course of fungal fermentation.

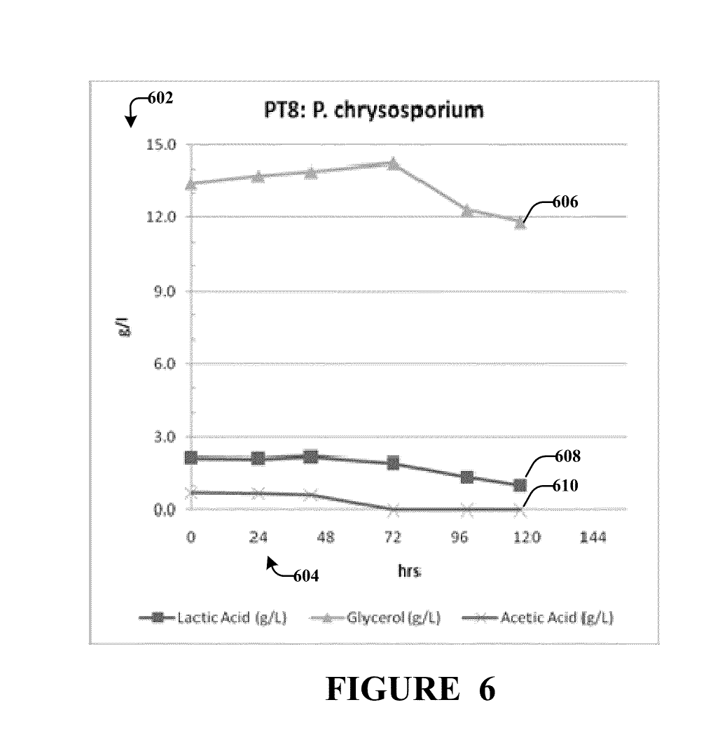

FIG. 6 illustrates the results for the strain P. chrysosporium. For this experiment, 20 liters of broth was fermented in a 30 liter fermentor vessel. The pH was maintained at 4.5 and temperature was maintained at 30 degrees Celsius. Samples of liquids and solids were taken each day over the fermentation period. Results from the analysis of the samples are illustrated with grams per liter of each component illustrated on the vertical axis, at 602, versus the length of fermentation, as indicated at 604. Glycerol 606, lactic acid 608 and acetic acid 610 are each plotted. For P. chrysosporium, the glycerol content increased slowly as a function of fermentation length until roughly 72 hours, after which the levels of glycerol appear to drop. As illustrated, acetic acid is reduced within the first 72 hours of fungal fermentation. Levels of lactic acid appear to slowly reduce over the course of fungal fermentation.

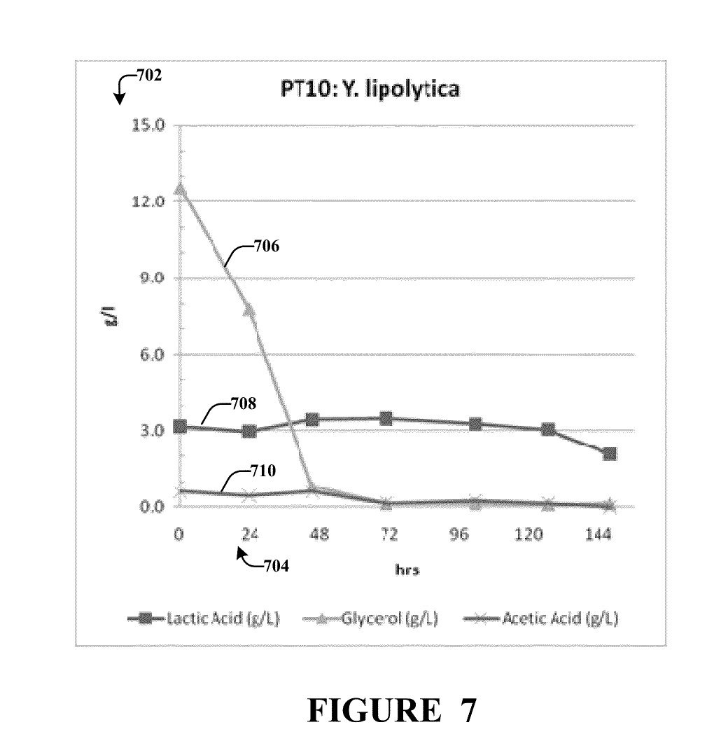

FIG. 7 illustrates the results for the strain Y. lipolytica. For this experiment, 20 liters of broth was fermented in a 30 liter fermentor vessel. The pH was maintained at 6.0 and temperature was maintained at 30 degrees Celsius. Samples of liquids and solids were taken each day over the fermentation period. Results from the analysis of the samples are shown with grams per liter of each component illustrated on the vertical axis, at 702, versus the length of fermentation, as indicated at 704. Glycerol 706, lactic acid 708 and acetic acid 710 are each plotted. For Y. lipolytica, the glycerol content decreases rapidly as a function of fermentation length, with the bulk of the glycerol consumed within 48 hours. As illustrated, acetic acid is reduced within the first 72 hours of fungal fermentation. Levels of lactic acid appear to slowly reduce after 96 hours of fermentation.

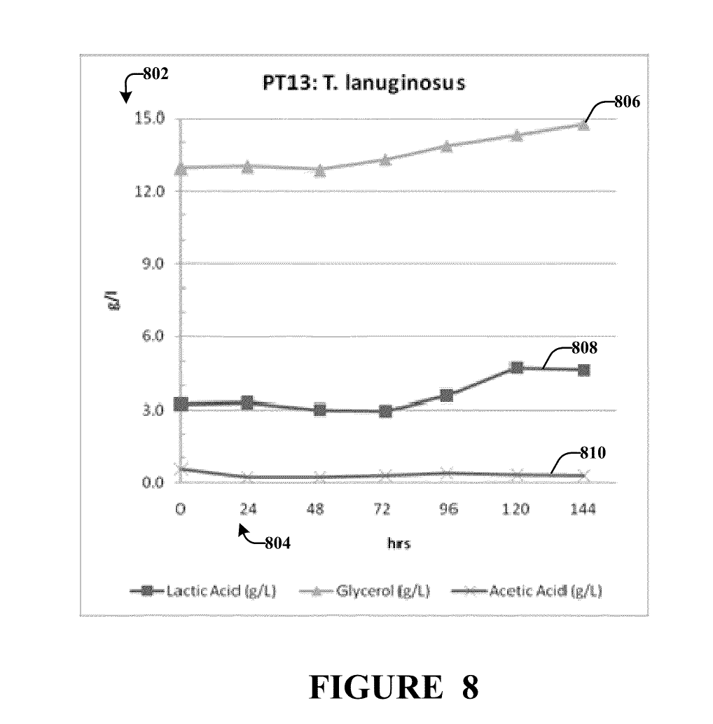

FIG. 8 illustrates the results for the strain T. lanuginosus. For this experiment, 20 liters of broth was fermented in a 30 liter fermentor vessel. The pH was maintained at 6.0 and temperature was maintained at 40 degrees Celsius. Samples of liquids and solids were taken each day over the fermentation period. Results from the analysis of the samples are shown with grams per liter of each component illustrated on the vertical axis, at 802, versus the length of fermentation, as indicated at 804. Glycerol 806, lactic acid 808 and acetic acid 810 are each plotted. For T. lanuginosus, the glycerol increases steadily after 48 hours of fermentation. Acetic acid levels appear to remain steady during fungal fermentation. Levels of lactic acid appear to increase after 72 hours of fermentation.

### Example 2

The solid samples then were analyzed to determine the variety and proportions of single-cell proteins for use as nutritional product. Successful solid fractions will have high total protein content and will contain beneficial amino acids, including: lysine, threonine, tryptophan, cystine and methionine. A mass balance study and a bioflo fermentation were also conducted to determine the amounts of each fraction created and to analyze individual components within each fraction (protein, fat, amino acid, fiber and starch).

For the mass balance the whole fermentation broth was used. The process flow diagram for the mass balance experiments are complex and consist of the following steps:

1) Obtain % solids of original thin stillage source by drying the remainder of thin stillage source at 50° C. and submitting for protein, fat, fiber and starch analysis.

2) Combine whole fermentation broth into carboy and continuously mix with large agitator. Then 5 replicate samples are collected for percent solids analysis.

3) Dry 1 liter of whole fermentation broth at 50° C. and submit solids for protein, fat, amino acid, fiber and starch analysis.

4) Prepare 5, 1-liter replicates to centrifuge. Initial weight was taken for the 1-liter replicates of whole fermentation broth. Samples were centrifuged at 4500×g for 10 minutes at 4° C. Then the centrate was poured off, weighed and saved for the fermentation studies. Finally, solids were collected. Five samples were taken for percent solids analysis and the remaining portion was dried at 50° C. Once dried the solid sample was submitted for protein, fat, amino acid, fiber and starch analysis.

Dried samples were prepared for protein, fat, starch and fiber analysis by grinding into a fine powder and placing into a 15 mL capped centrifuge tube with proper labels. A total of 100±5 mg of the ground sample was weighed into a tin foil cup and compressed into a pellet. The pellet then was placed into a rapid N cube elemental analyzer to determine total protein content. Leftover material then was prepared for amino acid analysis by digesting in 6 N HCl for 24 h at 110° C. After filtration and evaporation, the residue was dissolved in 3.2 pH citric acid buffer and 10 μL of this buffered solution was injected on a Dionex Bio-LC Ion Chromatography system with an AS-50 autosampler, AS50TC thermal compartment and GS-50 4 eluent gradient pump. The system is equipped with a 2×50 mm AminoPac PA-10 guard column and a 2×250 mm AminoPac PA-10 analytical column. The system is also equipped with a Chrome Tech Sensivate Post-Column reactor pumping ninhydrin reagent at 0.12 mL/min. The derivitized amino acids were visualized with a Dionex Variable Wavelength Detector at 570 nm and 440 nm wavelengths. A four-system eluent system was used, including 10 mM NaOH, 250 mM NaOH, 1 M NaOAc with 25 mM NaOH as a preservative and 100 mM Citric Acid as column cleaning agent. A complex gradient system was used to enact the separation.

The bioflo fermentation consisted of the following steps:

1) Sterilize 3.5 L of thin stillage in a bioflo 310 at 121° C. for 15 minutes. Following sterilization the thin stillage was cooled to 30° C. and was pH adjusted to 6.0 with NH₄OH. Airflow was set at 1.0 vvm and agitation was set at 450 rpm. Approximately 3 mL of a 5% w/w solution of antifoam (Foamblast, 55570) was added prior to starting the airflow.

2) Once stabilized the bioflo was inoculated with a sterile A. niger spore suspension to bring the inoculation level to 100,000 spores/mL.

3) The thin stillage was fermented for 5 days. Following fermentation the sample was centrifuged at 4500×g for 10 minutes at 4° C. The centrate was poured off, solids were taken, and the centrate was weighed. The solids fraction was also analyzed for percent solids and the remainder was dried at 50° C. Once dried the solid sample was submitted to Midwest Labs for a standard nutritional analysis, including: protein, fat, and fiber analysis. TABLE 1 and TABLE 2 illustrate the results of the mass balance profile for the Y. lipolytica and A. niger experiments. As illustrated, the protein content is increased from 19.80% in the original thin stillage to 37.05% in the Y. lipolytica solids. It is also interesting to note that Y. lipolytica uses the fat fraction as a food source decreasing the percentage from 21.24% in the thin stillage to 6.16% after fermentation. The A. niger results indicate that up to 58.84 dry g/L of fungal biomass can be obtained from a fermentation broth with a total solids content of 94.30 dry g/L.

FIG. 11 illustrates the protein content, at 1102, as a function of fermentation time, as indicated at 1104. Results indicate that the Y. lipolytica fungus produces the most favorable protein content reaching 41.95% after 6 days. The A. niger strain also significantly enhanced protein content to 35.13% when the fermentation pH was raised to 6.0. However, the P. chrysosporium strain did not produce favorable protein contents and only increased the final value to 23.49%.

The mass balance data also allowed for a commercially relevant calculation to determine how much of the single-cell protein product is produced. Looking at TABLE 4, using the data for centrifuged solids (g/L) the A. niger and Y. lipolytica strains will produce about 3.72 and 2.66 lbs of protein per bushel of corn processed.

Turning now to TABLE 3, a nutritional profile was also obtained from Midwest Labs to help quantify the usefulness of the fungal protein produced for A. niger. Total protein content and crude fat levels are of particular interest at 37.6% and 18.2% dw respectively.

### Example 3

The liquid samples where then analyzed to test the potential benefits of any extracellular enzymes captured in the liquid fraction. Successful backset sources will have reduced total and dissolved solids and will reduce enzyme loads or increase ethanol titers in raw starch ethanol fermentation. To test for suitability of using the treated thin stillage (fungal treated stillage) as a successful backset, corn flour was subjected to a standard simultaneous saccharification and fermentation (raw starch fermentation) in a 100 ml reactor at a solids loading level of 35% (w/v). The makeup water from each fungal source and RO water was used to bring the total mash to a volume of 70 mL. The fungal backset was included at 0%, 25% and 50%. Inactivated backset controls were also included by autoclaving at 121° C. for 15 minutes.

Samples were pH adjusted to 4.5 with 45% (w/w) KOH and 10% (v/v) H₂SO₄. Lactoside247 (192 oz/550,000 gal fermentor) and Urea (350 gal/550,000 gal fermentor) were added. An enzyme mixture for converting starch to sugar was then added at 500, 375, 250, 125, and 0 kg enzyme/550,000 gal mash to samples with an active backset. The enzyme mixture was also added at 500, 250, and 0 kg enzyme/550,000 gal mash to samples with an inactive backset. The reactors were incubated for 88 hours using standard temperature staging protocols. Samples were taken at 24 hour intervals and at 88 hours. At 88 hours, the residual starch, % solids and protein contents were also obtained.

FIG. 9 illustrates the impact of backset on ethanol titers and enzyme loading requirements. In this diagram, enzyme loading levels are indicated at 904. Ethanol titers are indicated at 902. The dark bar graph columns indicate fermentations which include a 50% treated thin stillage backset. The lighter column indicate fermentations which include a 25% treated thin stillage backset. The dotted line is a comparison to the fermentation using a water backset with a 250 kg enzyme loading level, and the solid horizontal line is a comparison to the fermentation using a water backset with a 500 kg enzyme loading level.

It can be clearly seen that the inclusion of a backset with treated thin stillage is beneficial to the fermentation process. With a 50% backset, the ethanol titers at 250 kg enzyme loading almost approach the ethanol titers of a fermentation performed with a water backset at 500 kg enzyme loading. At a 375 kg enzyme loading level both 25% and 50% backset outperform a water backset sample at 500 kg enzyme loading. In fact, at a 25% backset loading level the A. niger backset can reduce current enzyme loads by 25% and still increase ethanol titers by 0.4% (v/v), or standard enzyme levels can still be loaded and create a 0.63% (v/v) gain in ethanol titers This equates to a substantial reduction in enzyme usage (or increase in ethanol yield), which can result in a substantial cost savings for the ethanol production facility. Further, water usage may be significantly reduced as treated thin stillage makes up more of the fill backset.

### Example 4

Lastly, a study was performed to determine if the fungal treated backset (treated thin stillage) would provide any benefit to the saccharification of biomass typically utilized in cellulosic ethanol production facilities. For this study exploded second pass bale solids were added at 12% solids to each of 9-125 mL Erlenmeyer flasks with clarified thin stillage, A. niger backset, or P. chrysosporium backset included. The backset sources were added at 25% of the total makeup water and each source was tested in triplicate. Type III RO water was added to bring the final volume to 70 mL. Samples were pH adjusted to 5.5 with 45% (w/w) KOH and 10% (v/v) H₂SO₄ enzyme addition. The reactors then were loaded with cellulase enzymes at 6.0 mg protein per gram glucan content. Samples were saccharified at a temperature of 50° C. while shaking at 150 rpm for 96 hours. Samples were taken every 24 hours for HPLC analysis.

FIG. 10 illustrates the results of this analysis. In this example plot, theoretical glucose, at 1002, is compared against saccharification timing, as indicated at 1004. The backsets from both A. niger and P. chrysosporium were tested, but did not show any beneficial results for releasing glucose. However, the glucose release values still were similar to clarified thin stillage saccharifications, indicating that the treated thin stillage can be used to reduce the non-productive adsorption of enzymes in the biomass process.

The embodiments as disclosed and described in the detailed description (including the FIGURES and Examples) are intended to be illustrative and explanatory of the various aspects. Modifications and variations of the disclosed embodiments, for example, of the apparatus and processes employed (or to be employed) as well as of the compositions and treatments used (or to be used), are possible; all such modifications and variations are intended to be within the scope of the subject disclosure.

The word “exemplary” is used to mean serving as an example, instance, or illustration. Any embodiment or design described as “exemplary” is not necessarily to be construed as preferred or advantageous over other embodiments or designs, nor is it meant to preclude equivalent exemplary structures and techniques known to those of ordinary skill in the art. Rather, use of the word exemplary is intended to present concepts in a concrete fashion, and the disclosed subject matter is not limited by such examples.

The term “or” is intended to mean an inclusive “or” rather than an exclusive “or.” To the extent that the terms “comprises,” “has,” “contains,” and other similar words are used in either the detailed description or the claims, for the avoidance of doubt, such terms are intended to be inclusive in a manner similar to the term “comprising” as an open transition word without precluding any additional or other elements.

What is claimed is:

1. A system for improving stillage, comprising:
a bioreactor configured to receive stillage, wherein the bioreactor is further configured to receive an inoculation of a fungi, and wherein the bioreactor is further configured to ferment the fungi and stillage broth to generate a fungal biomass and a treated stillage, and further wherein the fungi is at least one of Aspergillus niger, Phanerochaete chrysosporium and Yarrowia lipolytica;
a separator configured to remove the fungal biomass from the treated stillage; and
a dryer configured to dry the fungal biomass.

2. The system of claim 1, wherein the fungi and stillage broth is maintained at a temperature at about 20 to 40° C.

3. The system of claim 1, wherein the fungi and stillage broth is maintained at a pH at about 4 to 6.

4. The system of claim 1, wherein the inoculation of the fungi includes at least one of inoculating spores and inoculating a cell culture.

5. The system of claim 1, further comprising piping configured to direct the treated stillage to a fermentation system as backset.

6. The system of claim 1, wherein the bioreactor is agitated.

7. The system of claim 1, wherein the bioreactor is aerated.

8. The system of claim 7, wherein the bioreactor is an airlift type bioreactor.

9. The system of claim 1, wherein the stillage includes whole stillage.

10. The system of claim 1, wherein the stillage includes thin stillage.

11. A method for improving stillage comprising:
receiving stillage;
inoculating the stillage with a fungi to generate a broth, wherein the fungi is at least one of Aspergillus niger, Phanerochaete chrysosporium and Yarrowia lipolytica;
fermenting the broth to generate a fungal biomass and a treated stillage;
removing the fungal biomass from the treated stillage; and
drying the fungal biomass.

12. The method of claim 11, wherein the fermenting comprises maintaining the broth at a temperature at about 20 to 40° C. during the fermenting.

13. The method of claim 11, wherein the fermenting comprises maintaining the broth at a pH at about 4 to 6 during the fermenting.

14. The method of claim 11, wherein the inoculating the stillage with the fungi comprises at least one of inoculating spores and inoculating a cell culture.

15. The method of claim 11, further comprising directing the treated stillage to a fermentation system as a backset.

16. The method of claim 11, wherein the fermenting comprises agitating the broth during the fermenting.

17. The method of claim 11, wherein the fermenting comprises aerating the broth during the fermenting.

18. The method of claim 17, wherein the fermenting comprises fermenting the broth in an airlift type bioreactor.

19. The method of claim 11, wherein the receiving comprises receiving stillage that comprises whole stillage.

20. The method of claim 11, wherein the receiving comprises receiving stillage that comprises thin stillage.
